(19) United States
(12) Patent Application Publication
(10) Pub. No.: US 2024/0111931 A1
(43) Pub. Date: Apr. 4, 2024

(54) TRIAL MANUFACTURING CONDITION PROPOSING SYSTEM AND TRIAL MANUFACTURING CONDITION PROPOSING METHOD

(71) Applicant: NGK INSULATORS, LTD., Nagoya-Shi (JP)

(72) Inventors: Yuki OKA HASHIMOTO, Kariya-Shi (JP); Fukunaga HIGUCHI, Nagoya-Shi (JP); Shingo SOKAWA, Okazaki-Shi (JP)

(73) Assignee: NGK INSULATORS, LTD., Nagoya-Shi (JP)

(21) Appl. No.: 18/539,491

(22) Filed: Dec. 14, 2023

Related U.S. Application Data
(63) Continuation of application No. PCT/JP2023/016025, filed on Apr. 21, 2023.

(30) Foreign Application Priority Data
Jun. 10, 2022 (JP) 2022-094726

(51) Int. Cl. G06F 30/27; G06F 30/17

(52) U.S. Cl. CPC G06F 30/27; G06F 30/17

(57) ABSTRACT

Trial manufacturing condition proposing system includes a characteristic evaluation data preprocessing unit, a feature value selection processing unit, a regression model creation processing unit, and a trial manufacturing condition proposing processing unit. The characteristic evaluation data preprocessing unit applies preprocessing to the characteristic evaluation data indicating an evaluation result of characteristics of the material. The feature value selection processing unit executes feature value selection processing on the characteristic evaluation data to which the preprocessing has been applied. The regression model creation processing unit executes regression model creation processing on the characteristic evaluation data, to which the preprocessing has been applied, based on the result of the feature value selection processing. The trial manufacturing condition proposing processing unit executes trial manufacturing condition proposing processing based on a regression model created by the regression model creation processing unit with respect to the characteristic evaluation data to which the preprocessing has been applied.

### TECHNICAL FIELD

The present invention relates to a system and method for proposing trial manufacturing conditions for a material(s) to material developers.

### BACKGROUND ART

In the field of material science to do research and development of materials, a method called “Materials Informatics (MI)” for efficiently predicting physical properties, structures, etc. of materials by using information technology (Informatics) such as statistical analysis and machine learning is widely used nowadays. Regarding the research and development of the materials by using this material informatics, for example, a technology of PTL 1 is known. PTL 1 discloses a system for estimating preparation conditions for substances having optimum physical properties and structures from a dataset including preparation conditions for each of a plurality of substances, which are samples, and substance information indicating physical properties and structures of the respective substances.

### CITATION LIST

### Patent Literature

PTL 1: WO2021/044913

### SUMMARY OF THE INVENTION

### Problems to be Solved by the Invention

At a site(s) of the material development using the material informatics, various kinds of evaluation tests for evaluating characteristics of materials, which are objects to be developed, are usually conducted and a computer is caused to execute processing for estimating preparation conditions, under which the relevant material will have good characteristics, based on data indicating the results of this evaluation test (hereinafter referred to as “characteristic evaluation data”). Under this circumstance, the characteristic evaluation data in a state of so-called raw data immediately after the results of the evaluation test are recorded often has dispersion in a distribution of the data or the scales of feature values are uneven. So, in such a case, it is necessary to perform processing for appropriately adjusting explanatory variables (hereinafter also referred to as “material design variables”) and objective variables, which are included in the characteristic evaluation data in a state of raw data, in advance (hereinafter referred to as “preprocessing”) in order to make it possible to execute subsequently processing normally.

However, generally, there are a considerable amount of the characteristic evaluation data which are to be execution objects of the preprocessing at the material development site(s) and there are also a wide variety of types of such characteristic evaluation data. Then, the system described in PTL 1 cannot perform the preprocessing on the characteristic evaluation data in the state of the raw data. Therefore, when trial manufacturing of a material is performed by using the technology described in PTL 1, it is necessary to manually perform extremely complicated preprocessing in advance in order to estimate the preparation conditions (hereinafter referred to as “trial manufacturing conditions”) and there is fear that the trial manufacturing conditions could not be estimated efficiently.

In light of the above-described problems, it is an object of the present invention to provide a technology capable of automatically executing the preprocessing of the characteristic evaluation data when proposing the trial manufacturing conditions for the material on the basis of the characteristic evaluation data.

### Means to Solve the Problems

A trial manufacturing condition proposing system according to the present invention is to propose trial manufacturing conditions for a material to a material developer and includes a characteristic evaluation data preprocessing unit, a feature value selection processing unit, a regression model creation processing unit, and a trial manufacturing condition proposing processing unit. The characteristic evaluation data preprocessing unit applies preprocessing to characteristic evaluation data indicating an evaluation result of characteristics of the material. The feature value selection processing unit executes feature value selection processing on the characteristic evaluation data to which the preprocessing has been applied. The regression model creation processing unit executes regression model creation processing on the characteristic evaluation data, to which the preprocessing has been applied, based on a result of the feature value selection processing. The trial manufacturing condition proposing processing unit executes trial manufacturing condition proposing processing based on a regression model created by the regression model creation processing unit with respect to the characteristic evaluation data to which the preprocessing has been applied.

A trial manufacturing condition proposing method according to the present invention is a method for proposing trial manufacturing conditions for a material to a material developer and is designed to cause a computer to execute: preprocessing on characteristic evaluation data indicating an evaluation result of characteristics of the material; feature value selection processing to be performed on the characteristic evaluation data to which the preprocessing has been applied; regression model creation processing to be performed on the characteristic evaluation data, to which the preprocessing has been applied, based on a result of the feature value selection processing; and trial manufacturing condition proposing processing to be performed based on a regression model created by the regression model creation processing with respect to the characteristic evaluation data to which the preprocessing has been applied.

Other than the above, the problems and their solutions which are disclosed by this application will be clarified by the section of DESCRIPTION OF EMBODIMENTS and descriptions of drawings.

### Advantageous Effects of the Invention

The preprocessing of the characteristic evaluation data can be performed automatically according to the present invention when proposing the trial manufacturing conditions for the material based on the characteristic evaluation data.

### BRIEF DESCRIPTION OF DRAWINGS

FIG. 1 is a schematic diagram illustrating the outline of a trial manufacturing condition proposing system according to one embodiment of the present invention;

FIG. 2 is a diagram illustrating functional blocks of the trial manufacturing condition proposing system according to one embodiment of the present invention;

FIG. 3 is a flowchart illustrating a flow of the entire processing of the trial manufacturing condition proposing system according to one embodiment of the present invention;

FIG. 4 is a flowchart illustrating the details of characteristic evaluation data preprocessing;

FIG. 5 is a diagram illustrating the outline of Box-Cox transformation processing;

FIG. 6 is a diagram illustrating the outline of standardization processing;

FIG. 7 is a flowchart illustrating the details of feature value selection processing;

FIG. 8 is a flowchart illustrating the details of regression model creation processing; and

FIG. 9 is a flowchart illustrating the details of trial manufacturing condition proposing processing.

### DESCRIPTION OF EMBODIMENTS

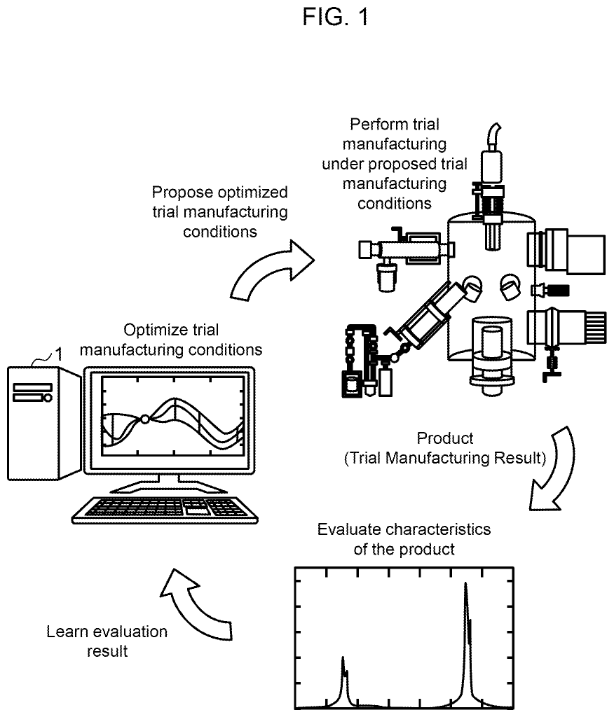

This embodiment will be described below in detail. FIG. 1 is a schematic diagram illustrating the outline of a trial manufacturing condition proposing system according to one embodiment of the present invention. A trial manufacturing condition proposing system 1 illustrated in FIG. 1 is designed to optimize each of various trial manufacturing conditions to be considered when performing trial manufacturing of a material, such as compositions of the material and firing conditions and to propose the results to a material developer who is a user of this system. This optimization of the trial manufacturing conditions is conducted by using various kinds of machine learning algorithms on the basis of characteristic evaluation data indicating the evaluation results of characteristics of the relevant material. Moreover, when doing so, various kinds of regression models such as Gaussian process regression (GPR), linear regression, regression trees (including a case by an ensemble method), regression by a neural network (Neural Network Regression), support vector regression (SVR), logistic regression, and LASSO regression (Least Absolute Shrinkage and Selection Operator Regression) are used as prediction models.

As illustrated in FIG. 1, when the user of the trial manufacturing condition proposing system 1 performs trial manufacturing of a material under trial manufacturing conditions proposed by the trial manufacturing condition proposing system 1 and evaluates characteristics of the material, which is a product of the trial manufacturing, data which indicates an evaluation value of the characteristics of this product is newly generated. After the trial manufacturing condition proposing system 1 is caused to learn this data as new characteristic evaluation data, the trial manufacturing condition proposing system 1 proposes more optimized trial manufacturing conditions to the user. Every time the user repeats this cycle, the trial manufacturing condition proposing system 1 according to this embodiment can propose the trial manufacturing conditions with a better predicted value of the characteristics. Incidentally, the trial manufacturing condition proposing system may include a function to perform trial manufacturing of the material and a function to evaluate the characteristics of the material which is the object of the trial manufacturing, and may be configured integrally with these functions.

The trial manufacturing condition proposing system 1 according to this embodiment is realized by one general-purpose computer device as illustrated in FIG. 1. The following explanation will be given by assuming that the trial manufacturing condition proposing system 1 is realized by one general-purpose computer device including one or more processor devices, one or more storage devices, one or more input/output devices, and wired or wireless communication lines connecting these components (either of which is not illustrated in the drawing).

This computer device is installed as, for example, a terminal inside a laboratory and is connected to various kinds of other terminals which are installed inside and outside the laboratory, various kinds of terminals such as laptop PCs, tablets, and smartphones owned by each user (hereinafter referred to as the “user's terminals”), and other equipment such as a server device(s) via a communication network such as the Internet 400 and dedicated lines. Incidentally, the computer device and the Internet 400 are connected by wire via well-known communication equipment (which is not illustrated in the drawing), but they may be connected wirelessly.

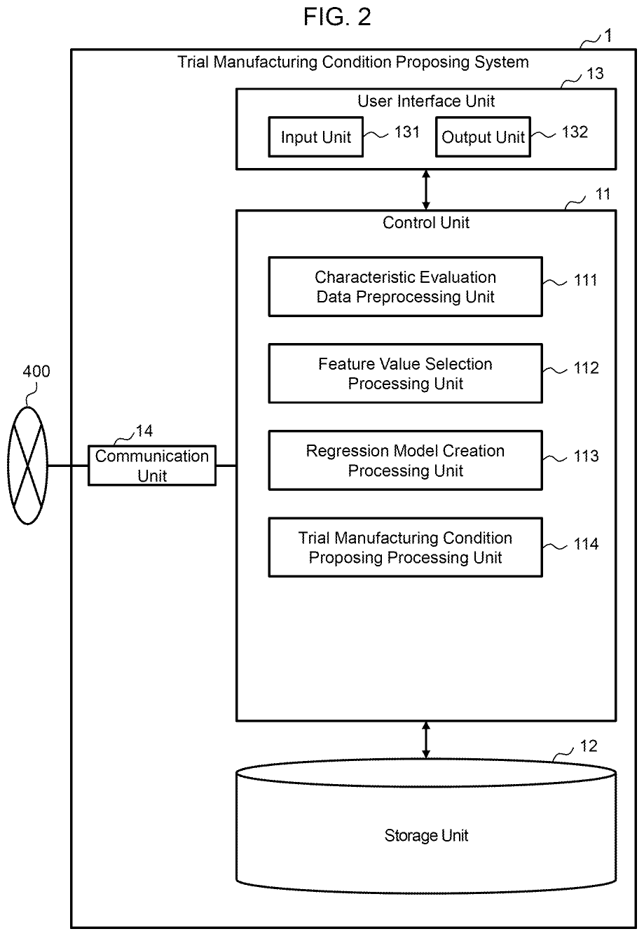

Next, an explanation will be provided about various kinds of functions included by the trial manufacturing condition proposing system 1 by referring to FIG. 2. FIG. 2 is a diagram illustrating functional blocks of the trial manufacturing condition proposing system according to one embodiment of the present invention. Incidentally, the respective blocks explained below indicate functional unit blocks, but not hardware unit components. The trial manufacturing condition proposing system 1 according to this embodiment is configured by including, as illustrated in FIG. 2, a control unit 11, a storage unit 12, a user interface unit 13, and a communication unit 14.

The control unit 11 executes various kinds of data processing based on the user's operation inputs detected by the user interface unit 13, data acquired by the communication unit 14, and programs and data which are stored in the storage unit 12. The control unit 11 also functions as an interface for the user interface unit 13, the communication unit 14, and the storage unit 12.

The control unit 11 has respective functional blocks of a characteristic evaluation data preprocessing unit 111, a feature value selection processing unit 112, a regression model creation processing unit 113, and a trial manufacturing condition proposing processing unit 114. The control unit 11 is configured by using, for example, processor devices such as a CPU (Central Processing Unit) and various kinds of co-processors (hereinafter also simply referred as “processors”) and can implement these functional blocks by executing specified programs. Incidentally, the control unit 11 may be configured by using, for example, logical circuits such as an FPGA (Field Programmable Gate Array), instead of the processors. Moreover, the control unit 11 may be configured by a combination of the processors and the logical circuits.

The programs to be executed by the control unit 11 may be installed from a program source(s). The program source(s) may be, for example, a program distribution computer(s), a recording medium/media which can be read by a computer(s), and so forth. Moreover, the programs executed by the control unit 11 may be configured by a device driver, an operating system, various kinds of application programs positioned in an upper layer thereof, and a library which provides common functions to these programs. Moreover, two or more programs may be implemented as one program and one program may be implemented as two or more programs.

The characteristic evaluation data preprocessing unit 111 applies preprocessing to the characteristic evaluation data in a state of so-called raw data immediately after it is recorded. This processing performed by the characteristic evaluation data preprocessing unit 111 will be referred to as characteristic evaluation data preprocessing.

The feature value selection processing unit 112 executes processing for selecting a feature value regarding an explanatory variable(s) with respect to the characteristic evaluation data to which the preprocessing has been applied. This processing executed by the feature value selection processing unit 112 will be referred to as feature value selection processing.

The regression model creation processing unit 113 executes processing for creating a regression model regarding the characteristic evaluation data, to which the preprocessing has been applied, based on the result of the feature value selection processing. This processing executed by the regression model creation processing unit 113 will be referred to as regression model creation processing.

The trial manufacturing condition proposing processing unit 114 executes processing for proposing trial manufacturing conditions for the material to the user on the basis of the regression model created by the regression model creation processing unit 113 with respect to the characteristic evaluation data to which the preprocessing has been applied. This processing executed by the trial manufacturing condition proposing processing unit 114 will be referred to as trial manufacturing condition proposing processing.

Incidentally, specific content of these processing sequences will be described later.

The storage unit 12 is configured by using, for example, storage devices such as a RAM(s) and a flash memory/memories and stores programs for supplying various kinds of processing instructions to the control unit 11 and data indicating various kinds of information to be used for the processing executed by the control unit 11. For example, the characteristic evaluation data to which the preprocessing has been applied by the characteristic evaluation data preprocessing unit 111 (hereinafter referred to as “preprocessed data”), data indicating regression models created by the regression model creation processing unit 113, and so forth are stored in the storage unit 12. The control unit 11 can implement the respective functional blocks of the characteristic evaluation data preprocessing unit 111, the feature value selection processing unit 112, the regression model creation processing unit 113, and the trial manufacturing condition proposing processing unit 114 mentioned earlier by reading/writing these pieces of information from/to the storage unit 12.

The user interface unit 13 is in charge of, besides accepting input operations from the user, processing relating to the user interface such as image display and sound output. The user interface unit 13 has respective functional blocks of an input unit 131 and an output unit 132. The input unit 131 detects various kinds of operations from the user. The input unit 131 is configured by using, for example, a keyboard, a pointing device, a touch panel, and so forth. The output unit 132 executes, for example, screen display and sound output for the user. The output unit 132 is configured by using, for example, a liquid crystal display and a touch screen.

The communication unit 14 is in charge of communication processing, which is performed via the Internet 400, with other equipment such as the user's terminals possessed by each user, the server device(s), and so forth. The communication unit 14 is configured by using, for example, an NIC (Network Interface Card) and an HBA (Host Bus Adapter).

This embodiment has been explained by describing that the respective functions of the trial manufacturing condition proposing system 1 are integrally implemented by one computer device. However, these respective functions may be implemented by a plurality of mutually connected computer devices or server devices. Also, the trial manufacturing condition proposing system 1 may be configured by including a general-purpose computer device such as a laptop PC, and a web browser which is installed in this general-purpose computer device or may be configured by including a web server and various kinds of portable equipment.

Moreover, the explanation about each function is an example and a plurality of functions may be put together as one function or one function may be divided into a plurality of functions.

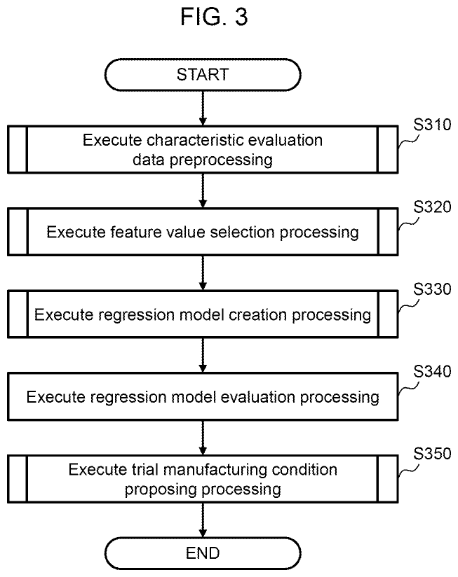

Next, an explanation will be provided about a flow of the entire processing of the trial manufacturing condition proposing system 1 with reference to FIG. 3. FIG. 3 is a flowchart illustrating a flow of the entire processing of the trial manufacturing condition proposing system according to one embodiment of the present invention. Incidentally, in the following explanation, there is a case where processing will be explained by referring to each function or program mentioned earlier as a subject; however, the processing explained by referring to the function or the program as the subject may be processing performed by a processor or a device having that processor.

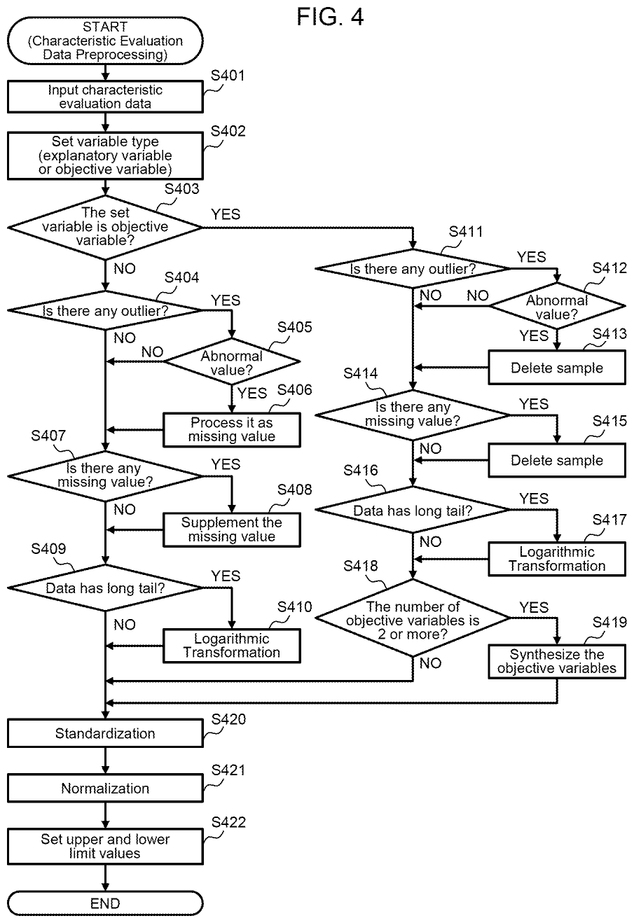

In step S310, the control unit 11 causes the characteristic evaluation data preprocessing unit 111 to execute the characteristic evaluation data preprocessing. Accordingly, the preprocessing is applied to the characteristic evaluation data, which then becomes the preprocessed data, so that it becomes possible to normally execute each subsequent processing. Incidentally, the details of the characteristic evaluation data preprocessing performed in step S310 will be explained later with reference to a flowchart in FIG. 4. When the characteristic evaluation data preprocessing is completed, the control unit 11 proceeds to step S320.

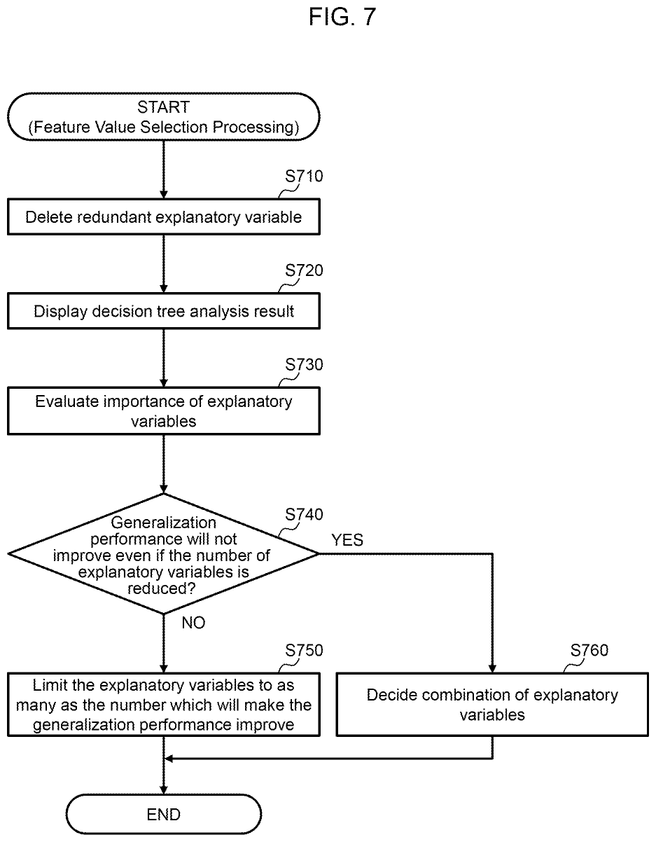

In step S320, the control unit 11 causes the feature value selection processing unit 112 to execute the feature value selection processing. Accordingly, a feature value regarding explanatory variables included in the preprocessed data is preferably selected in order to execute each subsequent processing. Incidentally, the details of the feature value selection processing performed in step S320 will be explained later with reference to a flowchart in FIG. 7. When the feature value selection processing is completed, the control unit 11 proceeds to step S330.

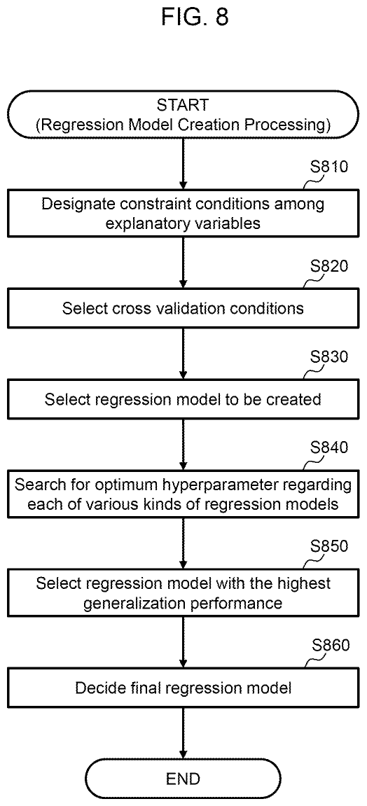

In step S330, the control unit 11 causes the regression model creation processing unit 113 to execute the regression model creation processing. Accordingly, a regression model(s) is created regarding the preprocessed data for which the feature value has been selected. Incidentally, the details of the regression model creation processing performed in step S330 will be explained later with reference to a flowchart in FIG. 8. When the regression model creation processing is completed, the control unit 11 proceeds to step S340.

In step S340, the control unit 11 executes the regression model evaluation processing. This regression model evaluation processing is to evaluate generalization performance which is an index indicating prediction accuracy of the relevant regression model with respect to each of the plurality of regression models created as a result of the respective processing sequences before and in step S330. This evaluation is conducted by performing, for example, cross validation with other regression models. The evaluation result is visualized by, for example, a graph such as a scatter diagram or a box-and-whisker diagram. As a result, the user can receive a proposal of the trial manufacturing conditions based on the regression model with good generalization performance. When the regression model evaluation processing is completed, the control unit 11 proceeds to step S350.

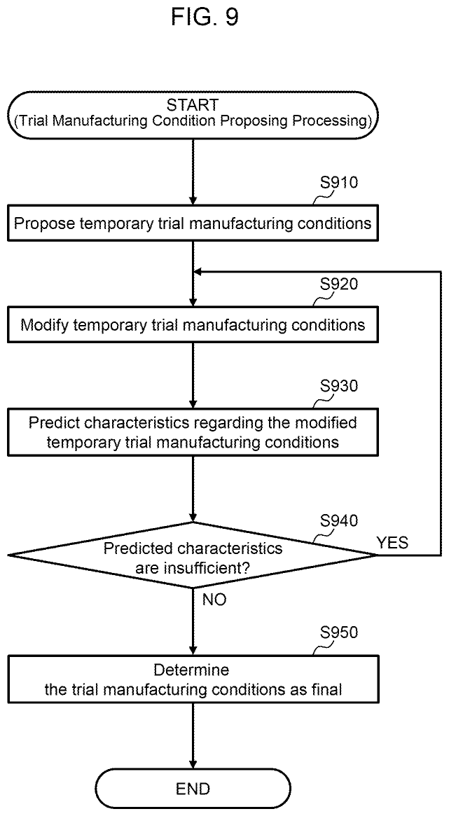

In step S350, the control unit 11 causes the trial manufacturing condition proposing processing unit 114 to execute the trial manufacturing condition proposing processing. With this trial manufacturing condition proposing processing, the user of the trial manufacturing condition proposing system 1 can modify the trial manufacturing conditions for the material, which have been proposed by the trial manufacturing condition proposing system 1, as appropriate to make them further preferable. The control unit 11 finds a predicted value of characteristics of the material if the material is to be trial manufactured under the trial manufacturing conditions modified by the user, by applying it to the selected regression model and presents the predicted value to the user. Specifically speaking, the user can interactively perform this work to modify the trial manufacturing conditions while checking the predicted value. Accordingly, the trial manufacturing condition proposing system 1 is designed as a system capable of incorporating the knowledge of the user, who is a developer of the relevant material, into the trial manufacturing conditions for the material to be proposed to the user. Incidentally, the details of the trial manufacturing condition proposing processing performed in step S350 will be explained later with reference to a flowchart in FIG. 9. When the trial manufacturing condition proposing processing is completed, the control unit 11 terminates the processing illustrated in the flowchart in FIG. 3 once.

The trial manufacturing condition proposing system 1 according to this embodiment executes each processing in steps S310 to S350 in FIG. 3 and proposes good trial manufacturing conditions to the user. Specifically speaking, with the trial manufacturing condition proposing system 1 according to this embodiment, in step S310, the necessary preprocessing is automatically applied to the characteristic evaluation data in the state of the raw data. So, each processing in steps S320 to S350 can be executed without manually performing the complicated preprocessing on the characteristic evaluation data. Moreover, with the trial manufacturing condition proposing system 1 according to this embodiment, the user can select a regression model with good generalization performance. Therefore, the trial manufacturing condition proposing system 1 can propose the trial manufacturing conditions, regarding which the predicted characteristic value is good, to the user.

Incidentally, as described earlier in relation to FIG. 1, after the user of the trial manufacturing condition proposing system 1 performs the trial manufacturing of the material under the trial manufacturing conditions proposed by the trial manufacturing condition proposing system 1, evaluates the characteristics of the product, and causes the trial manufacturing condition proposing system 1 to learn data indicating the evaluation result as new characteristic evaluation data, the user can then cause the trial manufacturing condition proposing system 1 to execute each processing in steps S310 to S350 in FIG. 3 again. In this case, the trial manufacturing condition proposing system 1 can propose more optimized trial manufacturing conditions to the user.

Specifically speaking, every time the trial manufacturing condition proposing system 1 according to this embodiment repeats each processing in steps S310 to S350 in FIG. 3 with respect to the same trial manufacturing object, it can propose the trial manufacturing conditions with a better predicted value of the characteristics.

FIG. 4 is a flowchart illustrating the details of the characteristic evaluation data preprocessing.

In step S401, the control unit 11 causes the characteristic evaluation data preprocessing unit 111 to receive an input of the characteristic evaluation data from the user via the input unit 131 or the communication unit 14. The characteristic evaluation data to be input to the trial manufacturing condition proposing system 1 may be, for example, category data, continuous data, or discrete data. Moreover, a specific data format of the characteristic evaluation data to be input to the trial manufacturing condition proposing system 1 can be decided as appropriate. When the processing in step S401 is completed, the control unit 11 proceeds to step S402.

In step S402, the control unit 11 causes the characteristic evaluation data preprocessing unit 111 to set a variable type with respect to the characteristic evaluation data whose input is received from the user in step S401. Under this circumstance, either an explanatory variable(s) or an objective variable(s) is set. The explanatory variable(s) is a variable which serves as the basis for finding a predicted value of the characteristics. In this embodiment, the composition of the material, firing conditions, etc. which constitute the trial manufacturing conditions correspond to the explanatory variables. Moreover, the objective variable(s) is a variable(s) which indicates a characteristic value(s) of the material to be trial manufactured, which becomes a prediction object. As an example of specific processing in step S402, the explanatory variable may be set as a default and a setting operation may be accepted from the user who wants to change it to the objective variable. When the processing in step S402 is completed, the control unit 11 proceeds to step S403.

In step S403, the control unit 11 causes the characteristic evaluation data preprocessing unit 111 to judge whether the variable which is set in step S402 is an objective variable or not. If it is not the objective variable (step S403: N), it is judged that the variable which is set in step S402 is an explanatory variable and the processing proceeds to step S404; and if it is judged that the variable which is set in step S402 is the objective variable (step S403: Y), the processing proceeds to step S411.

In step S404, the control unit 11 causes the characteristic evaluation data preprocessing unit 111 to judge whether or not there is any outlier with respect to the characteristic evaluation data to which the explanatory variable is set in step S402. This processing for judging whether any outlier exists or not is performed by, for example, indicating the characteristic evaluation data as a histogram and judging whether or not there is any value outside the range of an average value±2σ. If it is judged that there is an outlier (step S404: Y), the processing proceeds to step S405 to judge whether this outlier is an abnormal value or not; and if it is judged that there is no outlier (step S404: N), the processing proceeds directly to step S407.

In step S405, the control unit 11 causes the characteristic evaluation data preprocessing unit 111 to judge whether or not this outlier is an abnormal value with respect to the characteristic evaluation data to which the explanatory variable(s) is set and regarding which it is determined in step S404 that there is the outlier. This processing for judging whether it is an abnormal value or not is performed by, for example, judging whether or not there was any data input mistake upon the generation of the characteristic evaluation data and judging whether or not there is any failure of an evaluation test machine. If it is judged that the outlier is an abnormal value (step S405: Y), the processing proceeds to step S406 to make that abnormal value a missing value; and if it is judged that the outlier is not an abnormal value (step S405: N), the processing directly proceeds to step S407.

In step S406, the control unit 11 causes the characteristic evaluation data preprocessing unit 111 to make this abnormal value a missing value with respect to the characteristic evaluation data regarding which it is determined in step S405 that the outlier is the abnormal value. When the processing in step S406 is completed, the control unit 11 proceeds to step S407.

In step S407, the control unit 11 causes the characteristic evaluation data preprocessing unit 111 to judge whether or not there is a missing value regarding the characteristic evaluation data. If it is judged that there is a missing value regarding the characteristic evaluation data (step S407: Y), the processing proceeds to step S408 in order to supplement the missing value; and if it is judged that there is no missing value (step S407: N), the processing directly proceeds to step S409.

In step S408, the control unit 11 causes the characteristic evaluation data preprocessing unit 111 to supplement the missing value with respect to the characteristic evaluation data regarding which it is determined in step S407 that the missing value exists. The missing value supplementation processing is performed by, for example, using an average value, a median value, a minimum value, a maximum value, and so forth of the characteristic evaluation data, excluding the outlier(s), as values to supplement the missing value. Moreover, the missing value may be supplemented by means of linear interpolation. Furthermore, if the explanatory variable is not a continuous value, but is a category value, the missing value may be supplemented with an arbitrary character string. Incidentally, when the missing value is supplemented in step S408, the characteristic evaluation data preprocessing unit 111 may, for example, display the supplemented value in red letters in order to make it easier to identify the supplemented value. Moreover, the characteristic evaluation data preprocessing unit 111 may, for example, delete standards themselves without supplementing the relevant missing value in step S408. Furthermore, with the trial manufacturing condition proposing system 1 according to this embodiment, if a missing ratio of the explanatory variables is large, for example, if 50% or more of the data quantity is missing, the characteristic evaluation data preprocessing unit 111 can delete the relevant explanatory variables themselves. When the processing in step S408 is completed, the control unit 11 proceeds to step S409.

In step S409, the control unit 11 causes the characteristic evaluation data preprocessing unit 111 to express the characteristic evaluation data as a histogram in order to judge whether or not either one of tails of the characteristic evaluation data (a distribution range of characteristic values of all samples of the characteristic evaluation data in the histogram) is longer than a reference value. If it is judged that only either one of the tails of the characteristic evaluation data expressed as the histogram is longer than the reference value (step S409: Y), it means that the dispersion of the characteristic evaluation data is large, so the processing proceeds to step S410; and if it is judged that the tail of the characteristic evaluation data expressed as the histogram is shorter than the reference value (step S409: N), the dispersion of the characteristic evaluation data is sufficiently small to execute each subsequent processing, so the processing directly proceeds to step S420. Moreover, if it is judged that both the tails of the characteristic evaluation data expressed as the histogram are longer than the reference value (step S409: N) and if the histogram is in a distribution shape close to bilaterally symmetric, processing such as logarithmic transformation is not required, so that the processing directly proceeds to step S420. Incidentally, the characteristic evaluation data preprocessing unit 111 may execute the processing in step S409 to judge whether or not to perform the logarithmic transformation in step S410, for example, by using skewness as an index.

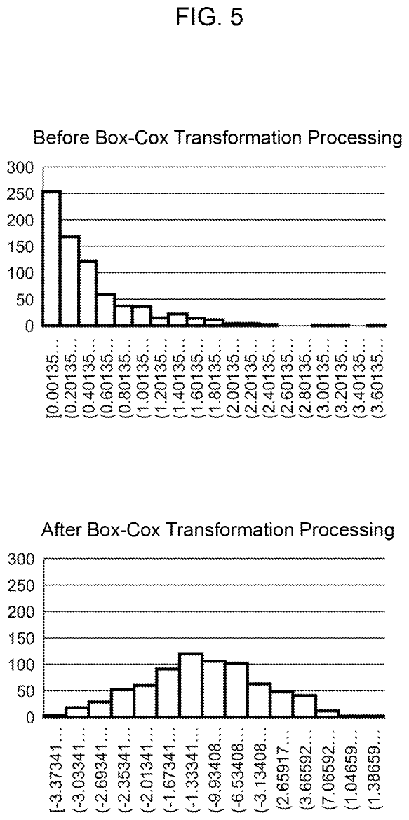

In step S410, the control unit 11 causes the characteristic evaluation data preprocessing unit 111 to perform the logarithmic transformation of a characteristic value of each sample of the characteristic evaluation data with respect to the characteristic evaluation data regarding which it is determined in step S409 that the tail(s) of the characteristic evaluation data when expressed as the histogram is longer than the reference value. This logarithmic transformation processing is performed by a method, for example, a Box-Cox transformation processing whose outline is illustrated in FIG. 5. As a result, the distribution of the characteristic evaluation data becomes closer to a normal distribution, so that the regression accuracy in the machine learning can be enhanced. Incidentally, with the trial manufacturing condition proposing system 1 according to this embodiment, the characteristic evaluation data preprocessing unit 111 automatically select an explanatory variable to which the logarithmic transformation processing is to be applied. However, the trial manufacturing condition proposing system may be configured so that the user can manually select the explanatory variable which should be an object of the logarithmic transformation processing. Moreover, for example, after applying the logarithmic transformation processing to the characteristic evaluation data in step S410, the characteristic evaluation data preprocessing unit 111 may visualize the degree of dispersion of the characteristic evaluation data by expressing the characteristic evaluation data before and after the logarithmic transformation processing as a histogram. When the processing in step S410 is completed, the control unit 11 proceeds to step S420.

In step S411, the control unit 11 causes the characteristic evaluation data preprocessing unit 111 to judge whether or not there is any outlier with respect to the characteristic evaluation data to which the objective variable is set in step S402. This processing for judging whether any outlier exists or not is performed in the same manner as in step S404 by, for example, expressing the characteristic evaluation data as a histogram. If it is judged that there is an outlier (step S411: Y), the processing proceeds to step S412 in order to judge whether this outlier is an abnormal value or not; and if it is judged that there is no outlier (step S411: N), the processing directly proceeds to step S414.

In step S412, the control unit 11 causes the characteristic evaluation data preprocessing unit 111 to judge whether or not this outlier is an abnormal value with respect to the characteristic evaluation data regarding which it is determined in step S411 that there is the outlier, and to which the objective variable is set. A specific example of the abnormal value includes a case in which, if explanatory variables are completely duplicate and there are standards with different characteristics, one of such explanatory variables may be the abnormal value. On one hand, if it is judged that it is an abnormal value (step S412: Y), the processing proceeds to step S413 in order to delete a sample relating to that abnormal value. This is because, if all the explanatory variables are duplicate and the standards with different characteristics are to be treated as abnormal values, it is necessary to delete such standards themselves unlike the case where one position of an explanatory variable attributable to an input mistake or the like is an abnormal value. On the other hand, if it is judged that it is not an abnormal value (step S412: N), the processing directly proceeds to step S414.

In step S413, the control unit 11 causes the characteristic evaluation data preprocessing unit 111 to delete the sample relating to this abnormal value with respect to the characteristic evaluation data regarding which it is determined in step S412 that the outlier is an abnormal value. Incidentally, if all the aforementioned explanatory variables are duplicate and there are standards with different characteristics, you may sometimes want to keep each one of the explanatory variables because the duplication might have some meaning. In that case, with the trial manufacturing condition proposing system 1 according to this embodiment, the processing from step S412 to S413 can be omitted. When the processing in step S413 is completed, the control unit 11 proceeds to step S414.

In step S414, the control unit 11 causes the characteristic evaluation data preprocessing unit 111 to judge whether or not there is any missing value with respect to the characteristic evaluation data. This is because there is sometimes a case in which the characteristic evaluation data may include a missing value(s) in advance. If it is judged that there is a missing value (step S414: Y), the processing proceeds to step S415 in order to delete a sample relating to the missing value; and if it is judged that there is no missing value (step S414: N), the processing directly proceeds to step S416.

In step S415, the control unit 11 causes the characteristic evaluation data preprocessing unit 111 to delete the sample relating to this missing value with respect to the characteristic evaluation data regarding which it is determined in step S414 that the missing value exists. When the processing in step S415 is completed, the control unit 11 proceeds to step S416.

In step S416, the control unit 11 causes the characteristic evaluation data preprocessing unit 111 to judge whether or not either one of tails of the characteristic evaluation data, which is expressed as a histogram, is longer than a reference value. If it is judged that only either one of the tails of the characteristic evaluation data expressed as the histogram is longer than the reference value (step S416: Y), it means that the dispersion of the characteristic evaluation data is large, so the processing proceeds to step S417; and if it is judged that the tail of the characteristic evaluation data expressed as the histogram is shorter than the reference value (step S416: N), the dispersion of the characteristic evaluation data is sufficiently small to execute each subsequent processing, so the processing directly proceeds to step S418. Moreover, if it is judged that both the tails of the characteristic evaluation data expressed as the histogram are longer than the reference value (step S416: N) and if the histogram is in a distribution shape close to bilaterally symmetric, processing such as logarithmic transformation is not required, so that the processing directly proceeds to step S418. Incidentally, the characteristic evaluation data preprocessing unit 111 may execute the processing in step S416 to judge whether or not to perform the logarithmic transformation in step S417, for example, by using skewness as an index.

In step S417, the control unit 11 causes the characteristic evaluation data preprocessing unit 111 to perform the logarithmic transformation, by a method such as the aforementioned Box-Cox transformation processing, with respect to the characteristic evaluation data regarding which it is determined in step S416 that one tail of the characteristic evaluation data when expressed as the histogram is longer than the reference value. As a result, the distribution of the characteristic evaluation data becomes closer to a normal distribution, so that the regression accuracy in the machine learning can be enhanced. When the processing in step S417 is completed, the control unit 11 proceeds to step S418.

In step S418, the control unit 11 causes the characteristic evaluation data preprocessing unit 111 to judge whether or not the number of objective variables which are set to the characteristic evaluation data is two or more. On one hand, if it is judged that the number of the set objective variables is two or more (step S418: Y), the processing proceeds to step S419 in order to synthesize all the set objective variables into one. On the other hand, if it is judged that the number of the set objective variable is not two or more (step S418: N), this means that the number of the set objective variables is one. In this case, it is unnecessary to perform the objective variable synthesis processing, so the processing directly proceeds to step S420. Incidentally, with the trial manufacturing condition proposing system 1 according to this embodiment, the characteristic evaluation data preprocessing unit 111 can execute each processing in step S420 to S422 also with respect to so-called multi-purpose characteristic evaluation data to which two or more objective variables are set. Therefore, when executing each subsequent processing with respect to such multi-purpose characteristic evaluation data, the characteristic evaluation data preprocessing unit 111 can omit the judgment processing in step S418.

In step S419, the control unit 11 causes the characteristic evaluation data preprocessing unit 111 to synthesize all the objective variables, which are set to the characteristic evaluation data, to one. This objective variable synthesis processing is performed by, for example, finding a weighted average of all the objective variables which are set to the characteristic evaluation data. As a result, the number of the objective variables which are set to the characteristic evaluation data becomes one, so that it becomes possible to execute processing for optimizing the trial manufacturing conditions, which should be then executed, as so-called single-purpose optimization. Incidentally, for example, if the optimization processing is performed via Bayesian optimization, the optimization processing can be executed even if a plurality of objective variables are set. So, in such a case, regarding the characteristic evaluation data which is judged in step S418 that the number of the set objective variable is two or more, subsequent optimization processing may be performed as the multi-purpose optimization while the number of the objective variables is kept as two or more, without performing the objective variable synthesis processing. When the processing in step S419 is completed, the control unit 11 proceeds to step S420.

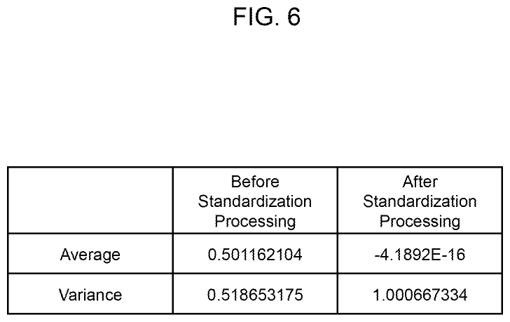

In step S420, the control unit 11 causes the characteristic evaluation data preprocessing unit 111 to perform standardization processing on the characteristic evaluation data as necessary. This standardization processing is to transform the scale of the characteristic evaluation data as its outline is illustrated in FIG. 6 so that an average=0 and a standard deviation (variance)=1 will be obtained. When the processing in step S420 is completed, the control unit 11 proceeds to step S421. Incidentally, if the normalization processing is applied to the characteristic evaluation data in step S421, the processing may omit the standardization processing in S420 and directly proceed to step S421.

In step S421, the control unit 11 causes the characteristic evaluation data preprocessing unit 111 to apply normalization processing to the characteristic evaluation data as necessary. This normalization processing is performed for the purpose of making it easier to compare between the explanatory variables with different scales. Incidentally, if the standardization processing is applied to the characteristic evaluation data in step S420, each subsequent processing may be executed without performing the normalization processing in S421. When the processing in step S421 is completed, the control unit 11 proceeds to step S422.

In step S422, the control unit 11 causes the characteristic evaluation data preprocessing unit 111 to set upper and lower limit values regarding the characteristic evaluation data. With the trial manufacturing condition proposing system 1 according to this embodiment, the characteristic evaluation data preprocessing unit 111 automatically extracts the upper and lower limit values from the existing data and sets them. Moreover, the user of the trial manufacturing condition proposing system 1 can manually modify the upper and lower limit values, which are automatically set by the characteristic evaluation data preprocessing unit 111, as necessary. When the processing in step S422 is completed, the control unit 11 stores the preprocessed data, which is the characteristic evaluation data to which the preprocessing is applied in steps S401 to S422 in FIG. 4, in the storage unit 12 and terminates the characteristic evaluation data preprocessing illustrated in the flowchart in FIG. 4.

FIG. 7 is a flowchart illustrating the details of the feature value selection processing.

In step S710, the control unit 11 causes the feature value selection processing unit 112 to acquire the preprocessed data from the storage unit 12 and judge whether or not any redundant explanatory variable(s) is included in the relevant preprocessed data. This judgment is performed with reference to whether or not a combination of explanatory variables with a correlation coefficient equal to or more than a specified number, for example, 0.8 or more. On one hand, if it is judged that any redundant explanatory variables are included in the relevant preprocessed data, the control unit 11 deletes one of the redundant explanatory variables and proceeds to step S720. Incidentally, with the trial manufacturing condition proposing system 1 according to this embodiment, the combination of the explanatory variables whose correlation coefficient is equal to or more than 0.8 is visualized for the user via the output unit 132, so that the explanatory variable to be deleted can be selected via the input unit 131. On the other hand, if it is judged that any redundant explanatory variable(s) is not included in the relevant preprocessed data, the control unit 11 directly proceeds to step S720.

Incidentally, in step S720, the control unit 11 causes the feature value selection processing unit 112 to perform decision tree analysis with respect to the preprocessed data, on which the processing in step S710 has been performed, and may also execute processing for visualizing the analysis result for the user via the output unit 132. Accordingly, the user can verify the validity of the judgment processing and the processing for deleting one of the redundant explanatory variable in step S710 on the basis of the visualized decision tree. When the processing in step S720 is completed, the control unit 11 proceeds to step S730.

In step S730, the control unit 11 causes the feature value selection processing unit 112 to execute processing for evaluating the importance with respect to the characteristic evaluation data regarding each of the plurality of explanatory variables included in the preprocessed data and automatically selecting a feature value, which is an explanatory variable of the regression model in the regression model creation processing performed in step S330, on the basis of the evaluation result. The trial manufacturing condition proposing system 1 according to this embodiment performs this processing for evaluating the importance of the explanatory variable(s) and selecting the feature value by a method of using a random forest regression tree called RFE (Recursive Feature Elimination) as an algorithm. This method is to remove the explanatory variables included in the preprocessed data one by one in ascending order of the importance and evaluate the explanatory variable(s), which is included in the relevant preprocessed data immediately before the prediction accuracy of a characteristic value obtained from the remaining explanatory variable(s) reduces to become equal to or lower than a threshold value, as an important variable(s). This method has an advantage of being capable of selecting a robust feature value and creating a model which can hardly cause overfitting, by using the random forest regression tree, which calculates the importance by combining a plurality of decision trees, as an algorithm. Therefore, the trial manufacturing condition proposing system 1 according to this embodiment is characterized in that the characteristic evaluation data has many explanatory variables as compared to the number of samples; and even when there is fear that overfitting might be caused if a characteristic prediction model is constructed from all the explanatory variables, it is possible to construct a model with high prediction accuracy by automatically selecting the feature value by this method. Moreover, the trial manufacturing condition proposing system 1 according to this embodiment can take the correlation with the objective variable(s) into consideration when performing the relevant processing. When the processing in step S730 is completed, the control unit 11 proceeds to step S740.

In step S740, the control unit 11 causes the feature value selection processing unit 112 to judge whether or not the generalization performance will not improve even if the number of the explanatory variables is reduced, with respect to the preprocessed data regarding which the importance of the explanatory variables is evaluated and the feature value is selected in step S730. If it is judged that the generalization performance will not improve even if the number of the explanatory variables is reduced (step S740: Y), the processing proceeds to step S760 in order to decide a combination of the explanatory variables; and if it is judged that the generalization performance will improve even if the number of the explanatory variables is reduced (step S740: N), the processing proceeds to step S750 in order to limit the explanatory variables to the number of explanatory variables whose generalization performance will improve.

In step S750, the control unit 11 causes the feature value selection processing unit 112 to perform processing for narrowing down the explanatory variables to the number of explanatory variables whose generalization performance will improve, with respect to the preprocessed data regarding which it is determined in step S740 that the generalization performance will improve when the number of the explanatory variable is reduced. With the trial manufacturing condition proposing system 1 according to this embodiment, this processing is performed by the feature value selection processing unit 112 by automatically selecting the explanatory variable(s) which will influence the improvement of the generalization performance, from among the explanatory variables included in the relevant preprocessed data. When the processing in step S750 is completed, the control unit 11 terminates the feature value selection processing illustrated in the flowchart in FIG. 7.

In step S760, the control unit 11 causes the feature value selection processing unit 112 to perform processing for deciding the number of the explanatory variables with respect to the preprocessed data regarding which it is determined in step S740 that the generalization performance will not improve even if the number of the explanatory variable is reduced. With the trial manufacturing condition proposing system 1 according to this embodiment, the user can decide the number of the explanatory variables. The feature value selection processing unit 112 decides the number of the explanatory variables, the input of which is accepted from the user via the input unit 131 or the communication unit 14. After the processing in step S760 is completed, the control unit 11 terminates the feature value selection processing illustrated in the flowchart in FIG. 7.

FIG. 8 is a flowchart illustrating the details of regression model creation processing.

In step S810, the control unit 11 causes the regression model creation processing unit 113 to set a constraint condition(s) among the explanatory variables with respect to the preprocessed data to which the feature value selection processing has been applied. With the trial manufacturing condition proposing system 1 according to this embodiment, for example, if the user wishes to impose a constraint so that a total of weight percent concentrations (wt %) of three kinds of raw materials A, B, and C would become 100% (A+B+C=100), the user can designate such condition as the constraint condition among the explanatory variables. The regression model creation processing unit 113 sets the constraint condition(s) among the explanatory variables, which is designated by the user via the input unit 131 or the communication unit 14. As a result, it is possible to prevent a proposal of the trial manufacturing conditions which are mismatching and unrealistic. When the processing in step S810 is completed, the control unit 11 proceeds to step S820.

In step S820, the control unit 11 causes the regression model creation processing unit 113 to select conditions to implement cross validation in order to evaluate a regression model to be created. Incidentally, the trial manufacturing condition proposing system 1 according to this embodiment evaluates each regression model by means of K-fold Cross Validation. Moreover, as its implementation condition, K=10 is set as a default value. In this case, the trial manufacturing condition proposing system 1 evaluates the regression model by means of 10-fold cross validation. Incidentally, with the trial manufacturing condition proposing system 1 according to this embodiment, the user can also select the cross-validation implementation condition(s).

Specifically speaking, the regression model creation processing unit 113 can accept the cross-validation implementation condition(s) from the user via the input unit 131 or the communication unit 14. When the processing in step S820 is completed, the control unit 11 proceeds to step S830.

In step S830, the control unit 11 causes the regression model creation processing unit 113 to select a candidate(s) for the regression model to be used as a prediction model for the search of the trial manufacturing conditions. When this happens, the regression model creation processing unit 113 selects, as the candidate(s), a regression model(s) regarding which it has accepted selection processing from the user via the input unit 131 or the communication unit 14. With the trial manufacturing condition proposing system 1 according to this embodiment, the user can select a plurality of regression models as the candidates from various kinds of regression models of, for example, Gaussian process regression, the aforementioned linear regression, regression trees (including a case by an ensemble method), regression via a neural network, support vector regression, logistic regression, and LASSO regression. When the processing in step S830 is completed, the control unit 11 proceeds to step S840.

In step S840, the control unit 11 causes the regression model creation processing unit 113 to search for and set an optimum hyperparameter for each of the various kinds of regression models selected as the candidates in step S830. With the trial manufacturing condition proposing system 1 according to this embodiment, with regard to each regression model, the regression model creation processing unit 113 automatically searches all the parameters; and when creating a regression model, it automatically sets a hyperparameter which would make that regression model acquire the best generalization performance. When the processing in step S840 is completed, the control unit 11 proceeds to step S850.

In step S850, the control unit 11 causes the regression model creation processing unit 113 to perform processing for selecting a regression model with the highest generalization performance from among the various kinds of regression models to which the optimum hyperparameters are set respectively. When the processing in step S850 is completed, the control unit 11 proceeds to step S860.

In step S860, the control unit 11 causes the regression model creation processing unit 113 to perform processing for deciding a final regression model. When the processing in step S860 is completed, the control unit 11 terminates the regression model creation processing illustrated in the flowchart in FIG. 8.

FIG. 9 is a flowchart illustrating the details of the trial manufacturing condition proposing processing.

In step S910, the control unit 11 causes the trial manufacturing condition proposing processing unit 114 to perform processing for searching for the trial manufacturing conditions based on the regression model created in step S850 in FIG. 8. The trial manufacturing condition proposing processing unit 114 executes this processing by means of optimization processing. Incidentally, the trial manufacturing condition proposing system 1 according to this embodiment is configured to be capable of using various kinds of optimization processing methods such as Mathematical Optimization (MO), Bayesian optimization (BO), Genetic Algorithm (GA), the Newton's Method (NM), and the Simplex Method (SM). As a result, the respective explanatory variables when a predicted value of the characteristics, that is, the objective variable becomes the best are proposed to the user as the temporary trial manufacturing conditions indicating the results of the relevant search processing. Moreover, under this circumstance, the trial manufacturing condition proposing processing unit 114 performs sensitivity analysis of the relevant temporary trial manufacturing conditions, evaluates the importance of the respective explanatory variables which constitute the relevant temporary trial manufacturing conditions, and presents the evaluation result together. Incidentally, if the used regression model is the Gaussian process regression, the trial manufacturing condition proposing system 1 according to this embodiment can select the trial manufacturing conditions which will maximize the acquisition function. When the processing in step S910 is completed, the control unit 11 proceeds to step S920.

In step S920, the control unit 11 causes the trial manufacturing condition proposing processing unit 114 to accept a modification(s) of the temporary trial manufacturing conditions, which have been proposed to the user in step S910, from the user. After accepting an input operation relating to the modification(s) of the values of the respective explanatory variables which constitute the temporary trial manufacturing conditions, the trial manufacturing condition proposing processing unit 114 modifies the temporary trial manufacturing conditions according to the modification content. When the processing in step S920 is completed, the control unit 11 proceeds to step S930.

In step S930, the control unit 11 causes the trial manufacturing condition proposing processing unit 114 to: find a predicted value of the characteristics of the material when the material is trial manufactured under the modified temporary trial manufacturing conditions by using the regression model; and present the calculation result to the user. Moreover, under this circumstance, the trial manufacturing condition proposing processing unit 114 also performs the sensitivity analysis of the modified temporary trial manufacturing conditions in the same manner as in step S910, evaluates the importance of the explanatory variables which constitute the relevant modified temporary trial manufacturing conditions, and presents the evaluation result together. Incidentally, this evaluation result is updated every time the user modifies the temporary trial manufacturing conditions; and the latest evaluation result is always presented to the user. When the processing in step S930 is completed, the control unit 11 proceeds to step S940.

In step S940, the control unit 11 causes the trial manufacturing condition proposing processing unit 114 to judge whether or not the predicted value of the characteristics as found in step S930 regarding the modified temporary trial manufacturing conditions is insufficient as a characteristic value of the material which is an object of the trial manufacturing. For example, if the regression model to be used is the Gaussian process regression, this judgment is performed with respect to each trial manufacturing condition by finding an acquisition function, which indicates an expected value of an improvement of the characteristics of the material which is trial manufactured under the relevant trial manufacturing condition, with respect to the modified temporary trial manufacturing condition and judging whether or not the difference between the value of the relevant acquisition function and a maximum value of the acquisition function is within a specified range. Incidentally, the acquisition function is calculated based on the predicted value μ of the characteristics of the material and a standard deviation σ indicating variations of the relevant predicted value when the trial manufacturing of the material is performed under an arbitrary trial manufacturing condition. If it is judged that the predicted characteristic value is insufficient (step S940: Y), the processing returns to step S920 and accepts an instruction to modify the trial manufacturing conditions from the user again; and if it is judged that the predicted characteristic value is not insufficient (step S940: N), this means that the predicted value of the characteristics of the material when performing the trial manufacturing of the relevant modified temporary trial manufacturing condition is sufficient, so that the processing proceeds to step S950. Specifically speaking, this processing in step S940 is performed repeatedly until it is judged that the predicted value of the characteristics of the material relating to the temporary trial manufacturing conditions is not insufficient.

In step S950, the control unit 11 causes the trial manufacturing condition proposing processing unit 114 to determine the temporary trial manufacturing conditions as final, regarding which it has been determined in step S940 that the predicted characteristic value is not insufficient, and propose them as the finalized trial manufacturing conditions to the user. When the processing in step S950 is completed, the control unit 11 terminates the trial manufacturing condition proposing processing illustrated in the flowchart in FIG. 9.

Incidentally, when the user performs the trial manufacturing of the material under the trial manufacturing conditions proposed in step S950 in FIG. 9 and the data indicating the evaluation result of the characteristics of the product is input as new characteristic evaluation data, the trial manufacturing condition proposing system 1 according to this embodiment proposes more optimized trial manufacturing conditions to the user on the basis of the newly input characteristic evaluation data as described above. In this case, the control unit 11 for the trial manufacturing condition proposing system 1 judges whether or not any missing value is included in the newly input characteristic evaluation data. On one hand, if it is judged that a missing value is included (Y), it is necessary to process this missing value as appropriate. Therefore, the characteristic evaluation data preprocessing is applied to the newly input characteristic evaluation data as follows: if this missing value relates to the explanatory variable(s), the processing is started from step S404 in FIG. 4; and if this missing value relates to the objective variable(s), the processing is started from step S411 in FIG. 4. On the other hand, if it is judged that any missing value is not included (N), it is unnecessary to perform the characteristic evaluation data preprocessing on the newly input characteristic evaluation data. Therefore, in such a case, the control unit 11 judges whether or not it is necessary to update the regression model. On one hand, if it is judged that it is necessary to update the regression model, the control unit 11 executes the feature value selection processing on the newly input characteristic evaluation data by starting the processing from step S420 in FIG. 4 in order to create a regression model again. On the other hand, if it is judged that it is unnecessary to update the regression model, the control unit 11 omits the feature value selection processing, the regression model creation processing, and the regression model evaluation processing and performs the trial manufacturing condition proposing processing on the newly input characteristic evaluation data by starting the processing from step S910 in FIG. 9 by using the regression model created last time. Incidentally, if it is unnecessary to update the regression model even when a missing value is included in the newly input characteristic evaluation data, the control unit 11 similarly omits the feature value selection processing, the regression model creation processing, and the regression model evaluation processing.

According to the above-described embodiment of the present invention, the following operational advantages can be achieved.

(1) The trial manufacturing condition proposing system 1 is a system for proposing trial manufacturing conditions for a material to a material developer and includes the characteristic evaluation data preprocessing unit 111, the feature value selection processing unit 112, the regression model creation processing unit 113, and the trial manufacturing condition proposing processing unit 114. The characteristic evaluation data preprocessing unit 111 applies preprocessing to characteristic evaluation data indicating an evaluation result of characteristics of the material (step S310). The feature value selection processing unit 112 executes feature value selection processing on the characteristic evaluation data to which the preprocessing has been applied (step S320). The regression model creation processing unit 113 executes regression model creation processing on the characteristic evaluation data, to which the preprocessing has been applied, based on a result of the feature value selection processing (step S330). The trial manufacturing condition proposing processing unit 114 executes trial manufacturing condition proposing processing based on a regression model created by the regression model creation processing unit with respect to the characteristic evaluation data to which the preprocessing has been applied (step S350). Consequently, the necessary preprocessing is automatically applied to the characteristic evaluation data in a state of raw data, so that each subsequent processing can be executed without manually performing complicated preprocessing on the characteristic evaluation data. Moreover, the trial manufacturing conditions with a good predicted characteristic value(s) can be proposed to the material developer who is the user by using the regression model with good generalization performance. Specifically speaking, if the trial manufacturing condition proposing system 1 according to this embodiment is employed, it is possible to derive good trial manufacturing conditions from an extremely wide variety of the trial manufacturing conditions, because of many kinds of feature values to be considered, on the basis of the characteristic evaluation data in a state of so-called raw data and propose them to the material developer.

(2) The preprocessing executed by the characteristic evaluation data preprocessing unit 111 includes at least one of the missing value supplementation processing (step S408), the outlier and abnormal value processing (steps S404 to S406 and steps S411 to S413), the standardization processing (step S420), or the normalization processing (step S421). Consequently, the necessary preprocessing is automatically applied to the characteristic evaluation data in the state of the raw data, which then becomes the preprocessed data, so that it becomes possible to normally execute each subsequent processing.

(3) The logarithmic transformation processing (step S410) executed by the characteristic evaluation data preprocessing unit 111 is the Box-Cox transformation processing (FIG. 5). Consequently, a distribution of the characteristic evaluation data can be made closer to a normal distribution, so that it is possible to enhance regression accuracy for the machine learning.

(4) The feature value selection processing unit 112 executes processing for: evaluating the importance of a plurality of explanatory variables included in the characteristic evaluation data, to which the characteristic evaluation data preprocessing unit 111 has applied the preprocessing, by using an arbitrary machine learning algorithm; and selecting an explanatory variable for the regression model from the plurality of explanatory variables based on the evaluation result (step S730). Consequently, the processing load of the control unit 11 can be reduced by reducing the number of the explanatory variables to the minimum number without causing degradation in the generalization performance. Also, as a result, it becomes possible to suppress overfitting and create a model with high regression accuracy even with respect to a small amount of data. Moreover, as a result, even if the user misses an important explanatory variable, it is possible to automatically pick up that explanatory variable.

(5) The above-mentioned machine learning algorithm is a random forest for calculating the importance by combining a plurality of decision trees. Consequently, it is possible to select a robust feature value and create a regression model which hardly causes overfitting.

(6) The regression model creation processing unit 113 executes processing for selecting a regression model with best generalization performance on the basis of a result of cross validation (step S850). Consequently, an appropriate regression model can be selected according to the content of the characteristic evaluation data.

(7) The regression model creation processing unit 113 sets an optimum hyperparameter to each created regression model (step S840). Consequently, regarding each regression model, the optimum hyperparameter is automatically set to the relevant regression model.

(8) The trial manufacturing condition proposing processing unit 114 executes processing for accepting a modification operation by the material developer with respect to the trial manufacturing conditions for the material, which are proposed to the material developer (step S920). Consequently, the material developer who is the user of the trial manufacturing condition proposing system 1 can incorporate their own knowledge into the modified trial manufacturing conditions by interactively performing the operation to modify the trial manufacturing conditions while checking a predicted value of the characteristics of the material when the trial manufacturing is performed under the trial manufacturing conditions before the modification.

(9) The trial manufacturing condition proposing processing includes the optimization processing. Consequently, it is possible to propose the trial manufacturing conditions, which will make the predicted value of the characteristics become the best, to the user.

Incidentally, the present invention is not limited to the above-described embodiment and can be implemented by using arbitrary constituent elements within the scope not departing from the gist of the invention.

The above-described embodiment and variations are merely examples and the present invention is not limited to their content unless the features of the invention are impaired. Moreover, various embodiments and variations are explained above, but the present invention is not limited to their content. Other aspects which can be thought of within the scope of technical ideas of the present invention are also included within the scope of the present invention.

### REFERENCE SIGNS LIST

1: trial manufacturing condition proposing system
11: control unit
12: storage unit
13: user interface unit
14: communication unit
111: characteristic evaluation data preprocessing unit
112: feature value selection processing unit
113: regression model creation processing unit
114: trial manufacturing condition proposing processing unit
131: input unit
132: output unit
400: Internet

1. A trial manufacturing condition proposing system for proposing trial manufacturing conditions for a material to a material developer,
the trial manufacturing condition proposing system comprising:
a characteristic evaluation data preprocessing unit that applies preprocessing to characteristic evaluation data indicating an evaluation result of characteristics of the material;
a feature value selection processing unit that executes feature value selection processing on the characteristic evaluation data to which the preprocessing has been applied;
a regression model creation processing unit that executes regression model creation processing on the characteristic evaluation data, to which the preprocessing has been applied, based on a result of the feature value selection processing; and
a trial manufacturing condition proposing processing unit that executes trial manufacturing condition proposing processing based on a regression model created by the regression model creation processing unit with respect to the characteristic evaluation data to which the preprocessing has been applied.

2. The trial manufacturing condition proposing system according to claim 1,
wherein the preprocessing executed by the characteristic evaluation data preprocessing unit includes at least one of missing value supplementation processing, outlier and abnormal value processing, standardization processing, or normalization processing.

3. The trial manufacturing condition proposing system according to claim 2,
wherein logarithmic transformation processing executed by the characteristic evaluation data preprocessing unit is Box-Cox transformation processing.

4. The trial manufacturing condition proposing system according to claim 1,
wherein the feature value selection processing unit executes processing for: evaluating importance of a plurality of explanatory variables included in the characteristic evaluation data, to which the characteristic evaluation data preprocessing unit has applied the preprocessing, by using an arbitrary machine learning algorithm; and selecting an explanatory variable of the regression model from the plurality of explanatory variables on the basis of a result of the evaluation.

5. The trial manufacturing condition proposing system according to claim 4,
wherein the machine learning algorithm is a random forest.

6. The trial manufacturing condition proposing system according to claim 1,
wherein the regression model creation processing unit executes processing for selecting a regression model with best generalization performance on the basis of a result of cross validation.

7. The trial manufacturing condition proposing system according to claim 1,
wherein the regression model creation processing unit sets an optimum hyperparameter to each created regression model.

8. The trial manufacturing condition proposing system according to claim 1,
wherein the trial manufacturing condition proposing processing unit executes processing for accepting a modification operation by the material developer with respect to the trial manufacturing conditions for the material, which are proposed to the material developer.

9. The trial manufacturing condition proposing system according to claim 1,
wherein the trial manufacturing condition proposing processing includes optimization processing.

10. A trial manufacturing condition proposing method for proposing trial manufacturing conditions for a material to a material developer,
the method designed to cause a computer to execute:
preprocessing on characteristic evaluation data indicating an evaluation result of characteristics of the material;
feature value selection processing to be performed on the characteristic evaluation data to which the preprocessing has been applied;
regression model creation processing to be performed on the characteristic evaluation data, to which the preprocessing has been applied, based on a result of the feature value selection processing; and
trial manufacturing condition proposing processing to be performed based on a regression model created by the regression model creation processing with respect to the characteristic evaluation data to which the preprocessing has been applied.
